(19) United States
(12) Patent Application Publication
(10) Pub. No.: US 2016/0206674 A1
(43) Pub. Date: Jul. 21, 2016

(54) FRUIT EXTRACTS AND EXTRACT FORMULATIONS OF CANARIUM ODONTOPHYLLUM AS ACTIVES AND RELATED INVENTION EMBODIMENTS

(71) Applicant: BIOTROPICS MALAYSIA BHD, Shah Alam, Selangor (MY)

(72) Inventors: MATTHIAS GEHLING, LEICHLINGEN (DE); ANNIE GEORGE, PUCHONG, SELANGOR (MY); SASIKALA M. CHINNAPPAN, PUCHONG, SELANGOR (MY); MAZRIA HASLINA BINTI MD AKIR, PADANG BESAR (U) PERLIS (MY)

(73) Assignee: BIOTROPICS MALAYSIA BHD, SHAH ALAMM SELANGOR (MY)

(21) Appl. No.: 14/913,953

(22) PCT Filed: Dec. 24, 2013
(86) PCT No.: PCT/MY2013/000258

§ 371 Date: Feb. 23, 2016

(51) Int. Cl. A61K 36/32; A61K 45/06

(52) U.S. Cl. CPC A61K 36/32; A61K 45/06

(57) ABSTRACT

Method for producing an extract or extract formulation of the fruit of Canarium odontophyllum comprising or consisting of the following steps:
(i) providing fruit material from Canarium odontophyllum, wherein said fruit material essentially consists of the endocarp of the seeds of Canarium odontophyllum,
(i-a) optionally drying the fruit material provided in step (i),
(ii) extracting the fruit material provided in step (i) or (i-a) with a mixture essentially consisting or consisting of water and an alcohol having 1 to 3 carbon atoms or consisting water and acetonitrile,
(iii) optionally partially or fully removing the alcohol having 1 to 3 carbon atoms of step (ii), and
   optionally adding an organic solvent of medium polarity to said aqueous residue and extracting the aqueous residue with said organic solvent or a supercritical solvent, such as supercritical carbon dioxide
      thereby obtaining an enriched extract,
(iv) optionally mixing the extract obtained in step (ii) or the enriched extract obtained in step (iii) with one or more solid carrier substances,
(v) optionally drying the enriched extract obtained in step (iii) or the mixture obtained in step (iv), preferably by spray-drying or freeze-drying., and related invention embodiments, especially for use in the treatment of PDE2 and/or PDE5 related diseases, and related invention embodiments.

The invention primarily relates to the use of certain extracts or extract formulations of certain parts of the fruits of Canarium odontophyllum as defined herein and their use as modulators of phosphodiesterase dependent conditions. Especially the modulation of phosphodiesterase5 (PDE5 or PDE-V) and phosphodiesterase2 (PDE2 or PDE-II) dependent conditions in the human body is described. The use (novel fields of application) for said extracts or formulations relates to the induction of vascular smooth muscle relaxation which can be modulated by PDE5 inhibition and that is beneficial for e.g. the improvement of pulmonary conditions, acute respiratory distress syndrome or male and female sexual dysfunction, or alternatively to fields of application that relate to PDE2 inhibition, e.g. sepsis, and cognition enhancement, or where both PDE2 and PDE5 are involved, to both types of treatment.

The present invention also relates to corresponding methods, to specific plant extracts and corresponding extract formulations obtainable from certain parts of the fruits of Canarium odontophyllum and to compositions, in particular pharmaceutical compositions, comprising an effective amount of such an extract or extract formulation.

A phosphodiesterase (PDE) is any enzyme that breaks a phosphodiester bond. Usually, people speaking of phosphodiesterase are referring to cyclic nucleotide phosphodiesterases, which have great clinical significance and are described below. However, there are many other families of phosphodiesterases. The cyclic nucleotide phosphodiesterases comprise a group of enzymes that degrade the phosphodiester bond in the second messenger molecules cAMP and cGMP. They regulate the localization, duration, and amplitude of cyclic nucleotide signaling within sub-cellular domains. PDEs are therefore important regulators of signal transduction mediated by these second messenger molecules.

A PDE5 (phosphodiesterase type 5) inhibitor is a compound (drug) used to block the degrading action of phosphodiesterase type 5 on cyclic GMP, e.g. in smooth muscle cells. These drugs are used in the treatment of erectile dysfunction. They were the first effective oral treatment available for the condition. Because PDE5 is also present in the arterial wall smooth muscle within the lungs, PDE5 inhibitors have also been explored for the treatment of pulmonary hypertension, a disease in which blood vessels in the lungs become overloaded with fluid, usually as a result of failure of the left ventricle of the heart.

References that are mentioned here in this respect (in particular regarding the desired pharmaceutical effects and fields of application connected to PDE2 (PDE-II) and PDE5 (PDE-V) inhibiting agents) are the following:

Human bronchus and pulmonary arteries are relaxed by theophylline and by selective inhibitors of PDE-III, while PDE-IV inhibitors also relax precontracted bronchus and PDE-V/I inhibitors relax pulmonary artery, see Eur. Respir. J. 1995, 8, 637-642.

Current Pharmaceutical Design, 2009, 15, 3521-3539 summarizes the effects of PDE5 inhibitors in non-urological conditions.

Future perspectives of PDE5 inhibitors are reported in Current Pharmaceutical Design, 2009, 15, 3540-3551.

Psychopharmacology 2009, 202, 419-443 gives an overview on selective PDE inhibitors as a promising target for cognition enhancement.

A review of herbal PDE inhibitors is given in Cytokine 2010, 49(2), 123-129. The scope of beneficial health improving fields of applications for PDE5 (PDE-V) inhibitors is given in Journal of the American College of Cardiology 2012, 59(1), 9-15.

Bryan G. Schwartz et al. (Journal of the American College of Cardiology, Volume 59, Issue 1, 2012, 9-15) described that PDE5 inhibitors (PDE5Is) improve erectile function by enhancing nitric oxide availability in the penis and its supplying vasculature, resulting in vasodilation and increased blood inflow. PDE5Is may show beneficial effects in cardiovascular diseases because phosphodiesterase-5 is also located elsewhere in the body, including the pulmonary and systemic vasculature and in hypertrophied myocardium. PDE5Is are approved for pulmonary arterial hypertension, given that they improved several hemodynamic and clinical parameters in large randomized trials. Initial evidence suggests that PDE5Is benefit patients with congestive heart failure and secondary pulmonary hypertension. PDE5Is seem to improve hemodynamic and clinical parameters in patients with high-altitude pulmonary edema (HAPE) and high-altitude pulmonary hypertension. In climbers with prior episodes of HAPE, PDE5Is prevented HAPE in 2 small randomized trials. In small randomized trials of PDE5Is, patients with Raynaud's phenomenon demonstrated improved blood flow, fewer symptoms and frequency of attacks, and resolution of digital ulcers. In addition to enhancing vasodilation, PDE5Is seem to protect the myocardium through complex pathways that involve nitric oxide, cyclic guanosine monophosphate, protein kinase G, extracellular-signal-regulated kinase, B-cell lymphoma protein-2, and Rho kinase inhibition. In animal models of acute myocardial infarction, PDE5 has consistently reduced infarct size indicating cardioprotection and PDE5Is also promote reverse remodeling and reduce myocardial apoptosis, fibrosis, and hypertrophy. PDE5Is might also benefit patients with treatment-resistant hypertension, preeclampsia, or peripheral arterial disease. This review presents the pathophysiology and trial data with regard to the use of PDE5Is for cardiac diseases.

There exists a need for effective and toxicological safe products, in particular of natural origin, that support, promote, and preferably improve or ameliorate cognitive performance.

US 2011/0151033 A1 reports that certain compounds and extracts comprising said compounds obtainable from plants of genera from the family of Lamiaceae, especially from the genus Orthosiphon, are useful as cognition enhancers.

US 2007/0161628 relates especially to novel stereospecific derivatives of 2,3-benzodiazepine type as inhibitors of phosphodiesterases, especially PDE2 and PDE4, and uses thereof in the therapeutic field.

Several documents disclose Ginkgo biloba extracts and certain constituents thereof inter alia exhibit phosphodiesterase inhibiting activity (see e.g. FR 2865652, WO 01/19381, WO 2005/004858, WO 2005/004890 and WO 2012/063198).

U.S. Pat. No. 6,395,313 B1 discloses oil obtained from the nuts of several varieties of Ngali Nut trees. The three most common varieties of Ngali Nut Trees are Canarium Indicium, Canarium Solomonesis and Canarium Harveyi. The Ngali Nut Tree is harvested to remove the Ngali Fruit. To extract the nut, the skin from the Ngali Fruit is removed and the in-shell nuts dried. The nuts are then cracked open to extract the kernel which is then pressed to extract the Ngali Nut Oil.

WO 2005/053718 relates to a method for the treatment or prophylaxis of a condition selected from a non-arthritic inflammatory condition, an arthritic condition, an aesthetic skin condition and a muscle injury in a subject, said method comprising administering to said subject oil from a Canarium nut or other part of a Canarium plant or horticultural relative thereof or one or more pharmacologically active fractions, extracts or components thereof or a formulation comprising the oil or an active fraction, extract or component thereof, wherein the inflammatory condition is treated by the administration of an active fraction, extract or component of the oil.

PI 20085159 discloses a liquid oil and/semi-solid extract of pulp and skin mixture from Canarium odontophyllum, and potential uses in various food preparations and applications. It also can be used as natural source for antioxidant, as food preservative and additives. It is explicitly mentioned that the pulp (flesh) and skin of the fruits are the parts of the fruits that are used for the production of the claimed vegetable oil. Claimed beneficial antioxidant activity was especially high in the fruit parts skin, skin/pulp and pulp. Up to 52% of the pulp comprises of fat. The pulp of the fruit together with the skin was separated from the seed before preparation of the vegetable oil.

PI 2010005011 discloses a vegetable fat/oil from Canarium odontophyllum pulp (copo), a food article or food material that is excellent in maintaining healthy blood cholesterol levels and improvement of antioxidant status. Pulp, peel and the inner soft part of the kernels is used as the starting material for the preparation of the disclosed oil. In order to obtain the inner soft/white part of the kernels, the hard woody endocarp (kernel shell) was manually broken.

PI2010005584 relates to an edible semi-solid/solid oil formulation from Canarium odontophyllum kernel oil (coko) improving plasma lipid profile and total antioxidant status. The kernel oil is obtained by first soaking mature fruits with hot water, second separating the peel and pulp from the seed and third manually breaking the woody endocarp (kernel shell) in order to obtain the fatty white/cream waxy inner part for oil preparation.

WO 2011/122927 relates to edible oil having high polyphenolic content compared to virgin olive oil and palm oil. Disclosed and claimed are liquid oil and/semi-solid extract of pulp and skin mixture from Canarium odontophyllum, and potential uses in various food preparations and applications. Additionally, they can be used in various food applications as source of fat in the form of liquid or semi-solid. WO 2011/122927 further relates to antioxidant activity and capacity of both pulp and flesh of Canarium odontophyllum that is highly correlated to the polyphenolic content of the oil extract. Further, the disclosed and claimed materials can be used as natural source for antioxidant, as food preservative and additives as well as part of food ingredient in normal food preparation and preparation of various commercial products and used as base in pharmaceutical and cosmetic formulations.

International Food Research Journal 2010, 319-326, and Journal of Biomedicine and Biotechnology, Vol. 2010, Article ID 871379, 8 pages, doi:10.1155/2010/871379 (http://dx.doi.org/10.1155/2010/871379) investigated and report on the antioxidant properties of skin, flesh and kernel of Canarium odontophyllum fruit.

In Food Analytical Methods DOI: 10.1007/s12161-011-9250-0, the antioxidant capacity of extracts from defatted dabai (Canarium odontophyllum) fruit is described. The peel of a defatted dabai fruit extracted using methanol contained a high total phenolics and Trolox equivalent antioxidant capacity (TEAC). Higher total phenolics and TEAC values were observed in water extract of the defatted dabai peel than ethanol, acetone, and ethyl acetate extracts. Hence, the use as a natural antioxidant was proposed for a methanol extract of a defatted dabai peel. Major phenolics in defatted dabai peel extracted using methanol were catechin and epigallocatechin while in water extract, major phenolic acid was ellagic acid.

In Food Chemistry, 124: 1549-1555, and Food Analytical Methods. 5: 339-350, and Journal of Food Composition and Analysis. 23: 777-781 antioxidant capacity of Canarium odontophyllum fruits is described. It has been is correlated to carotenoids and phenolic content.

Journal of Food Composition and Analysis 2011, Volume 23, pages 772-776 and Volume 24 pages 670-677 describe the nutritional composition and antioxidant properties of Canarium odontophyllum fruits of different districts in Malaysia. Lipid was the major macronutrient in dabai fruits, while the predominant minerals were calcium, sodium and potassium. The fruit protein was rich in aspartic and glutamic acids. Purple dabai fruits from Kapit were found to contain the highest total phenolic levels, flavonoids and anthocyanin contents and to exhibit the most significant antioxidant activities. Antioxidant activities were highly correlated with total phenolic and flavonoid contents of dabai fruits.

Effect of defatted Dabai (Canarium odontophyllum Miq.) pulp ingestion on lipid peroxidation and antioxidant status of hypercholesterolemia-induced rabbits was reported at the 4^{th} International Conference on the Development of Biomedical Engineering in Vietnam, 08-12 Jan. 2012, IFBME Proceeding, Volume 40; 137-140.

In Food Analytical Methods 2012, 5(1), 126-137 the phenolic compounds of dabai fruits from different divisions of Sarawak are identified and quantified. The fruit variety affected the anthocyanidins and anthocyanins profile, but had little or no effect on the phenolic acids and flavonoids profile of the fruits.

Evidence-Based Complementary and Alternative Medicine, Vol. 2012, Article ID 838604, 10 pages, doi:10.1155/2012/838604 reports an antiatherosclerotic effect of Canarium odontophyllum fruit parts in rabbits fed high Cholesterol diet. The pulp was separated by peeling it off from pit (the inner part of fruit). The kernel was obtained by crushing the pit. Then, the pulp and kernel were freeze-dried and ground before undergoing oil extraction. Among the Canarium odontophyllum fruit parts, defatted pulp was associated with the greatest reduction in atherosclerotic plaque formation, induced by a significant reduction in low-density lipoprotein cholesterol, total cholesterol, and lipid peroxidation levels. The presence of high dietary fiber and high levels of antioxidants with potent activity was essential factor contributing to the retardation of atherosclerosis and a reduction in coronary artery disease risk. Consequently, these results indicate the potential use of Canarium odontophyllum defatted pulp as a hypocholesterolemic and antioxidative agent apart from its ability to slow the progression of atherosclerosis.

Oxidative Medicine and Cellular Longevity, Vol. 2012 (2012), Article ID 840973, reported on protective effect of pulp oil extracted from Canarium odontophyllum fruit on blood lipids, lipid peroxidation, and antioxidant status in rabbits. Supplementation of Canarium odontophyllum pulp oil or kernel oil resulted in favorable changes in blood lipid and lipid peroxidation with enhancement of SOD, GPx, and plasma TAS levels. These changes showed that oils of Canarium odontophyllum could be beneficial in improving lipid profile and antioxidant status as when using part of normal diet. The oils can be used as alternative to present vegetable oil.

Asian Journal of Biochemistry 2012, Vol. 7, Issue: 2, 80-89 reported cholesterol-lowering and atherosclerosis inhibitory effect of Canarium odontophyllum defatted pulp in cholesterol fed rabbits. Significant reduction in total cholesterol and low-density lipoprotein cholesterol together with increment in high-density lipoprotein cholesterol relative to normal diet fed rabbits was demonstrated. Furthermore, the atherosclerotic plaque formation was diminished. The material that was used for this study was the defatted pulp of Canarium odontophyllum fruit. The defatted pulp was prepared by separating the pulp and he kernel, freeze drying the pulp and defatting the dry pulp material with solvent (Chloroform Methanol=2:1 v/v).

Journal of Food Composition and Analysis; Vol. 23, Issue 8, December 2010, 772-776 reports on the fatty acid composition, vitamin E contents and physicochemical properties of Canarium odontophyllum pulp and kernel oils. Use as vegetable oil for cooking is recommended.

Up to this date, there is no study done to evaluate the full potential of extracts prepared from the endocarp of the seeds of Canarium odontophyllum especially for food and/or functional or pharmaceutical uses.

The primary object of the present invention was to provide alternative active substances and compositions that inhibit at least one phosphodiesterase (PDE), preferably PDE5 or PDE2, and based on said inhibiting activity can induce vascular smooth muscle relaxation (PDE5) or improve of pulmonary conditions (PDE5), male and female sexual dysfunction (PDE5) or improve acute respiratory distress syndrome (PDE5) or enhance cognition (PDE2) in mammals, preferably in human beings. Additionally, said substances should preferably be naturally occurring compounds.

It has now been found that this primary object can be achieved by using certain extracts or extract formulations obtainable from certain fruit parts of Canarium odontophyllum.

In a first aspect, the present invention relates to a method for producing an extract or extract formulation of the fruit of Canarium odontophyllum comprising or consisting of the following steps:
(i) providing fruit material from Canarium odontophyllum, wherein said fruit material essentially consists of the wooden endocarp of the pyrene of Canarium odontophyllum,
(i-a) optionally drying the fruit material provided in step (i),
(ii) extracting the fruit material provided in step (i) or (i-a) with a mixture essentially consisting of or consisting of water and an alcohol having 1 to 3 carbon atoms or consisting of water and acetonitrile, preferably with a mixture of ethanol and water, wherein the total volume ratio (v/v) of said alcohol:water is in the range of 1:30 to 30:1, preferably in the range of 1:10 to 10:1, more preferably in the range of 1:5 to 5:1, even more preferably in the range of 1:2 to 2:1, and yet more preferably in the range from 1.2:1 to 1:1.2, or most preferably in the range of 1:1,
(iii) optionally partially or fully removing the alcohol having 1 to 3 carbon atoms or acetonitrile of step (ii), preferably at a temperature below 80° C., more preferably in the from 15 to 60° C., thereby obtaining an aqueous residue, and
   optionally adding an organic solvent of medium polarity to said aqueous residue and extracting the aqueous residue with said organic solvent or a supercritical solvent, such as supercritical carbon dioxide or, wherein the organic solvent is e.g. selected from the group consisting of n-butanol, iso-butanol, 2-butanone, 3-pentanone, methyl n-propyl ketone, methyl iso-propyl ketone, methyl isobutyl ketone, methyl isoamyl ketone, methyl-t-butyl ether, methyl acetate, ethyl acetate, propyl acetate, butyl acetate, pentyl acetate, ethyl propanoate and ethyl butanoate or a mixture thereof, and subsequently isolating the organic layer comprising the organic solvent of medium polarity comprising the enriched extract of interest;
   thereby obtaining an enriched extract,
(iv) optionally mixing the extract obtained in step (ii) or the enriched extract obtained in step (iii) with one or more carrier substances, preferably one or more solid carrier substances selected from the group consisting of maltodextrins, silica, talc, lactose, sorbitol, mannitol, dextrose, sucrose, starches, gum acacia, calcium phosphate, orally acceptable stearate salts, preferably magnesium stearate, alginates, tragacanth, gelatins, calcium silicates, cellulose and cellulose derivatives, preferably amorphous cellulose, microcrystalline cellulose or methyl cellulose, polyvinylpyrrolidones, and propylhydroxybenzoates, and
(v) optionally drying the enriched extract obtained in step (iii) or the mixture obtained in step (iv), preferably by evaporation of solvent, spray-drying or freeze-drying.

Surprisingly, it now has been found that the extracts or extract formulations according to the present invention (as defined herein) can inhibit one or more phosphodiesterases, in particular PDE5 and PDE2. Thus, the extracts or extract formulations according to the present invention are useful in the prophylactic and/or therapeutic treatment of conditions related to the modulation of phophodiesterase 5 (PDE5 or PDE-V) and phosphodiesterase 2 (PDE2 or PDE-II) in the mammal body, especially in the human body. Fields of application for said extract preparations relate to the therapeutic and/or prophylactic treatment to achieve induction of vascular smooth muscle relaxation which can be modulated by PDE5 inhibition and that is beneficial for e.g. the improvement of pulmonary conditions, acute respiratory distress syndrome, or in the case of male and female sexual dysfunction, while use fields of application that relate to PDE2 inhibition are e.g. sepsis, and cognition enhancement.

The extracts or extract formulations according to the present invention were shown in own experiments to be potent inhibitors of phosphodiesterases, in particular of PDE5 and PDE2, and thus can particularly be used in the prophylactic and/or therapeutic treatment of conditions related to induction vascular smooth muscle relaxation (PDE5) or improvement of pulmonary conditions (PDE5) or male and female sexual dysfunction (PDE5), or for prophylactic and/or therapeutic treatment of (especially in order to improve it) acute respiratory distress syndrome (PDE5), or in order to enhance cognition (PDE2) in mammals, preferably in human beings.

This is particularly unexpected since extracts obtained from the seeds or the pulp/peel of fruits of Canarium odontophyllum in own experiment showed weaker phosphodiesterase inhibiting effects than the extract formulations according to the present invention.

The genus Canarium belongs to the family of Burseraceae, which contains 18 genera with roughly 550 species. The genus Canarium comprises ca. 75 species, some of which have great economic relevance; Canarium ovarium or Canarium indicum for example are among to the most important nut-bearing trees in South-East Asia and Australia. Scientific synonyms are Canarium beccarii, Canarium multifidum, Canarium palawanense. Ethnobotanical and other names are Dabai, Kembayau, Bundui-bundui, Dabang, Dabu, Dawai, Kambayau, Kedongdong, Kumbayan, Kurihang, Saluan, Sibu olive tree.

In addition to the well-documented use of the fruits of various Canarium species as food, decoctions of the fruits are used in China as sedatives and antiphlogistics. Dried seeds are used as contraceptives in South Korea and Papua New Guinea. There are various other traditional uses documented, e.g. the resin of Canarium schweinfurthii is used to treat diarrhoea and dysentery in parts of Africa. Interestingly, a decoction of the dried stem bark of Canarium schweinfurthii is used to treat diabetes in Philippines.

Canarium odontophyllum is a large evergreen tree, up to 36 m in height, with alternate pinnate leaves, which are toothed at the margins. The tree carries white-yellow flowers of ca. 10 mm diameter in panicles. The fruits are about 35 mm long purple-black drupes (also called “Sibu Olive” or “Dabai”) of an oily appearance, with the edible, unctuous mesocarp surrounding three angular seeds. Canarium odontophyllum is native to Borneo, Sumatra and the Philippines. It is usually growing in undisturbed lowland forests up to an altitude of ca. 600 m. The trees can be found mainly on limestone soil, mostly on hills and regions, occasionally also in swamps and along rivers. In secondary forests, it is usually present as a pre-disturbance remnant tree.

The fruits of Canarium odontophyllum are drupes. They have a three-locular ovary with a terminal stigma that develops as a three-sided fruit with a single-seeded pyrene (kernel, stone), i.e. the pyrene contains only one seed.

In the context of the present invention it is essential that the plant material used comprised of the endocarp of Canarium odontophyllum, a certain part of the fruit of Canarium odontophyllum.

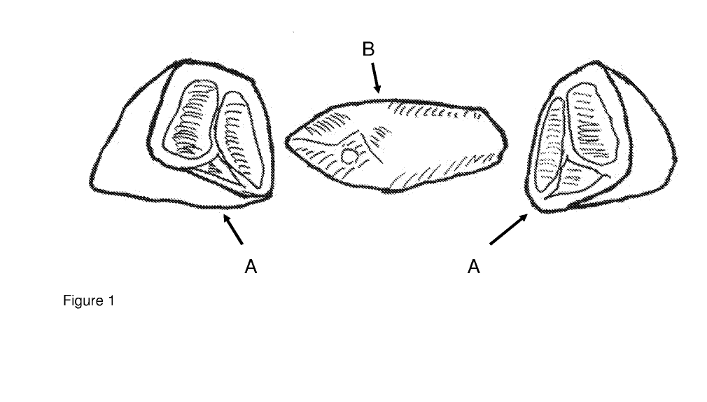

FIG. 1 is a diagrammatic view of a pyrene (kernel, stone) of a fruit of Canarium odontophyllum split open in halves. With reference to FIG. 1, the two different parts forming the pyrene (stone, kernel) are indicated: woody endocarp (A) and waxy solid seed (B). The woody endocarp (A) is shown after having split the pyrene into two halves. Inside of the endocarp (A) is a three angular waxy semi-solid seed (B).

In the context of the present invention, essential or essentially, e.g. “essentially consists of” or “essentially consisting of” or “essential amount”, means especially that the total weight share is 90 wt. % or more, preferably 95 wt. % or more, more preferably 98 wt. % or more, most preferably 99 wt. % or more, based on the total amount used. For example, “fruit material essentially consisting of the seedless woody endocarp” means that especially the total amount of seedless woody endocarp is 90 wt. % or more, preferably 95 wt. % or more, more preferably 98 wt. % or more, most preferably 99 wt. % or more, based on the total amount of fruit material employed.

In the context of the present invention, “essentially free of” means that the total weight share is 10 wt. % or less, preferably 5 wt. % or less, more preferably 2 wt. % or less, most preferably 1 wt. % or less, based on the total amount used.

In the context of the present invention, a therapeutic or pharmaceutical use or method is considered as medical treatment, including prophylaxis.

In summary, the extracts or extract formulations according to the present invention, in particular in one of the preferred embodiments defined herein, are active substances or mixtures of substances that inhibit at least one phosphodiesterase (PDE), preferably PDE5 or PDE2, and based on said inhibiting activity can induce vascular smooth muscle relaxation (PDE5) or improve of pulmonary conditions (PDE5), male and female sexual dysfunction (PDE5) or improve acute respiratory distress syndrome (PDE5) or enhance cognition (PDE2) in mammals, preferably in human beings.

Additionally, said substances are preferably naturally occurring compounds.

“Obtainable” means that a product (e.g. extract or extract formulation) may be obtained by a specifically described method or other methods leading to the product, or preferably that it is obtained by said method.

Where ratios or percentages are given, these refer to the weight (e.g. percent by weight, wt. %), unless indicated otherwise. Where volume ratios (v/v) are given, these refer to the volumes at 25° C. and 1013 mbar.

“Comprising” or “including” wherever used herein is meant not to be limiting to any elements stated subsequently to such term but rather to encompass one or more further elements not specifically mentioned with or without functional importance, that is, the listed steps, elements or options need not be exhaustive. In contrast, “consisting of” would be used where the elements are limited to those specifically after “consisting of”.

By the term “extract”, either a direct extract (in liquid or preferably dried form), e.g. obtained as described below, or preferably a further enriched extract (obtainable e.g. by one or more further purification steps after extraction, e.g. chromatography, for example as described below) is meant.

By “administered” or “administering” herein is meant administration of a prophylactically and/or therapeutically effective dose of an extract formulation according to the present invention, to a human being in need of such treatment. By “effective amount” or “effective dose” herein is meant an amount or a dose that produces the (therapeutic) effects for which it is administered, especially a smooth muscle relaxation effect or any other PDE2- or PDE5-modulation mediated effect mentioned herein.

A “patient” or “subject” for the purposes of the present invention relates to a mammal, especially a human being. Thus, extract formulations according to the present invention are applicable to both humans and mammals. In the preferred embodiment the patient is a human. The patients will be treated either in prophylactic or therapeutic intention.

The terms “dry”, “dried form”, “dry weight” and the like refer to matter (such as an extract, a composition etc.) essentially free of or completely free of water and essentially free of or completely free of organic solvents, in particular being free of water and free of substances having a boiling point of less than 300° C. at 1013 mbar.

The terms “liquid” and “solid” refer to the state of matter, e.g. a compound, carrier or composition, at 25° C. and 1013 mbar.

It should be stressed that constituents (including active components) are not uniformly distributed in a particular plant, i.e. not all individual parts of a plant contain all constituents occurring in said plant. Additionally, the proportions and ratios of the different constituents occurring in a particular plant vary strongly from one plant part to another. As a consequence, the properties, in particular the activity and effects of the different plant parts or extracts obtained from the different plant parts is not foreseeable.

In a preferred method according to the present invention, the fruit material provided in step (i) is essentially free of Canarium odontophyllum seeds. In a preferred method according to the present invention, the fruit material provided in step (i) is free of Canarium odontophyllum seeds and/or pulp and/or peel.

In a preferred method according to the present invention the fruit material provided in step (i) essentially consists or consists of endocarp of the fruits of Canarium odontophyllum. To illustrate which parts of the fruit Canarium odontophyllum constitute the endocarp reference is made to the accompanying FIG. 1 (FIG. 1).

For the sake of clarity, it is noted that in the context of the present invention not whole fruits of Canarium odontophyllum are used, and preferably the peel, the pulp and the seeds, are excluded from the fruit material employed in the methods, uses, and extract formulations according to the present invention.

FIG. 1 is a diagrammatic view not to scale of a dried pyrene (stone, kernel) of a fruit of Canarium odontophyllum cut in halves, thereby uncovering the actual seed.

With reference to FIG. 1, the two main parts forming the pyrene are indicated: the woody endocarp (including the seed coat) (A), and the seed (B). The hatched areas of the (in reality dark brown) woody endocarp (A) in FIG. 1 represent the three loculi, i.e. the hollow parts of the woody endocarp after removing the seed (A). In an intact pyrene, one loculus of the woody endocarp encloses the dark brown seed (A).

Part (A) as depicted in FIG. 1 is also referred to as “seedless woody endocarp of Canarium odontophyllum” in the context of the present invention. After drying, the weight ratio of the total amount of seedless woody endocarp (including the seed coat) (A) to the total amount of seed (B) is about 83:17.

The extracts or extract formulations according to the present invention or produced according to a method of the present invention are particularly useful as active substances or substance mixtures that inhibit at least one phosphodiesterase (PDE), preferably PDE5 or PDE2, and based on said inhibiting activity can be used for prophylactic and/or therapeutic treatment to induce vascular smooth muscle relaxation (PDE5) or improve pulmonary conditions (PDE5), or in the case of male and female sexual dysfunction (PDE5), or for the prophylactic and/or therapeutic treatment of (especially in order to improve it) acute respiratory distress syndrome (PDE5) or (prophylactically and/or therapeutically) in order enhance cognition (PDE2) in mammals, preferably in human beings. Additionally, said substances are preferably naturally occurring compounds.

The fruit material may be used without prior treatment or after treatment, such as drying, milling or the like. Prior to performing the extraction step(s), the fruit material is preferably comminuted, e.g. via chopping, milling or grinding or combinations thereof.

Preferably, fresh (i.e. not dried) fruit material of Canarium odontophyllum is used in the extraction step(s).

The extract or extract formulation according to the present invention or produced according to a method of the present invention may be prepared by any extraction method known in the art, however, with the proviso that certain extraction parameters are observed (as mentioned above), in particular by using the specific part of the fruit of Canarium odontophyllum as indicated in the context of the present invention.

In a further embodiment of the invention, the extraction can be followed by a step for further enrichment, e.g. solvent partition (liquid/liquid—extraction). In a preferred method using solvent partition, an aqueous extract residue—optionally filled up with additional water—is partitioned between a hydrophilic aqueous phase and a hydrophobic phase, preferably a solvent or solvent mixture of medium polarity forming a separate phase in the presence of water, preferably comprising or consisting of one or more esters, ethers, ketones, or alcohols, wherein the solvents used each have at least 4 carbon atoms. The organic phase comprises the extract of interest in the invention embodiments.

Additionally or alternatively, the extracts may also be subject to chromatographic enrichment steps, e.g. preparative high performance chromatography.

The extraction can be carried out at lower or elevated or ambient temperature, e.g. in the range from −20° C. to the (actual) boiling point of the solvent or solvent mixture employed, e.g. from ambient temperature (about 20° C.) to said boiling point. The extraction may be improved by moving the solvent and/or the plant material, e.g. by stirring, or by ultrasound, or by milling and/or chopping during extraction, or the like.

Auxiliary means such as (especially ultrasonic) sonication, warming/heating, stirring, re-extraction, evaporation or the like, may be used to allow for appropriate extraction, enrichment and purification.

Additional further processing of the (enriched) extracts used to obtain an extract formulation according to the present invention is possible, e.g. by filtering (e.g. through paper, sintered glass, charcoal (also allowing for decoloration) or silica).

The extraction of the fruit material is preferably carried out with a mixture essentially consisting or consisting of water and ethanol, wherein the total volume ratio (v/v) of ethanol:water is in the range of 1:30 to 30:1, preferably in the range of 1:10 to 10:1, more preferably in the range of 1:5 to 5:1, even more preferably in the range of 1:2 to 2:1, and yet more preferably in the range of 1.2:1 to 1:1.2, especially 1:1, preferably at a temperature below 75° C., more preferably in the range from 15 to 60° C., particularly preferably in the range from 20 to 50° C.

A preferred method according to the present invention for producing an extract or extract formulation of the endocarp of the fruit of Canarium odontophyllum comprises or consists of the following steps:
(i) providing fruit material from Canarium odontophyllum, wherein said fruit material essentially consists of the wooden endocarp of the pyrene of Canarium odontophyllum,
(ii) extracting the fruit material provided in step (i) with a mixture essentially consisting of or consisting of water and ethanol, wherein the total volume ratio (v/v) of ethanol:water is in the range of 1:5 to 5:1, preferably in the range of 1:2 to 2:1, and yet more preferably in the range of 1.2:1 to 1:1.2, especially 1:1,
(iii) partially or fully removing the ethanol of step (ii), preferably at a temperature below 80° C., more preferably in the range from 15 to 60° C., thereby obtaining an aqueous residue, and
   optionally adding an organic solvent of medium polarity to said aqueous residue and extracting the aqueous residue with said organic solvent or a supercritical solvent, such as supercritical carbon dioxide, wherein the organic solvent is preferably selected from the group consisting of n-butanol, iso-butanol, 2-butanone, 3-pentanone, methyl n-propyl ketone, methyl iso-propyl ketone, methyl isobutyl ketone, methyl isoamyl ketone, methyl-t-butyl ether, methyl acetate, ethyl acetate, propyl acetate, butyl acetate, pentyl acetate, ethyl propanoate and ethyl butanoate or a mixture thereof, and subsequently isolating the organic layer comprising the organic solvent of medium polarity comprising the enriched extract of interest, thereby obtaining an enriched extract,
(iv) optionally mixing the enriched extract obtained in step (iii) with one or more solid carrier substances, preferably one or more solid carrier substances, e.g. selected from the group consisting of maltodextrins, silica, talc, lactose, sorbitol, mannitol, dextrose, sucrose, starches, gum acacia, calcium phosphate, orally acceptable stearate salts, preferably magnesium stearate, alginates, tragacanth, gelatins, calcium silicates, cellulose and cellulose derivatives, preferably amorphous cellulose, microcrystalline cellulose or methyl cellulose, polyvinylpyrrolidones, and propylhydroxybenzoates,
(v) drying the enriched extract obtained in step (iii) or the mixture obtained in step (iv), preferably by evaporation of solvent, spray-drying or freeze-drying.

A further preferred method according to the present invention for producing an extract or extract formulation of the endocarp of the fruit of Canarium odontophyllum comprises or consists of the following steps:
(i) providing fruit material from Canarium odontophyllum, wherein said fruit material essentially consists of the wooden endocarp of the pyrene of Canarium odontophyllum,
(ii) extracting the fruit material provided in step (i) with a mixture consisting of water and ethanol, wherein the total volume ratio (v/v) of ethanol:water is in the range of 1:2 to 2:1, and yet more preferably in the range from 1.2:1 to 1:1.2, or and preferably in the range of 1:1,
(iii) removing an essential amount or the total amount of ethanol of step (ii), preferably at a temperature below 80° C., more preferably in the range from 15 to 60° C., thereby obtaining an aqueous residue, and
   optionally adding an organic solvent of medium polarity to said aqueous residue and extracting the aqueous residue with said organic solvent or a supercritical solvent as supercritical carbon dioxide, wherein the organic solvent is selected from the group consisting of methyl acetate, ethyl acetate, propyl acetate, butyl acetate or a mixture thereof, and subsequently isolating the organic layer comprising the organic solvent of medium polarity comprising the enriched extract of interest,
   thereby obtaining an enriched extract,
(iv) optionally mixing the enriched extract obtained in step (iii) with one or more solid carrier substances, preferably one or more solid carrier substances, e.g. selected from the group consisting of maltodextrins, silica, lactose, sorbitol, mannitol, starches, gum acacia, calcium phosphate, magnesium stearate, alginates, tragacanth, gelatins, calcium silicates, cellulose and cellulose derivatives, preferably amorphous cellulose, microcrystalline cellulose or methyl cellulose, polyvinylpyrrolidones, and propylhydroxybenzoates,
(v) drying the enriched extract obtained in step (iii) or the mixture obtained in step (iv), preferably by evaporation of solvent, spray-drying or freeze-drying.

The weight ratio of the total fruit material to the total amount of aqueous alcoholic solvent used in the extraction preferably is in the range from 2:1 to 1:5, more preferably in the range from 1:1 to 1:3, even more preferably in the range from 1:1 to 1:2.

Preferably, the extracts can subsequently be further enriched by one or more additional purification steps, such as liquid-liquid-distribution, precipitation or chromatography, or combinations of two or more thereof.

In another aspect, the present invention relates to an extract or extract formulation, preferably in solid form, obtainable by a method comprising or consisting of the following steps:
(a-i) providing fruit material from Canarium odontophyllum, wherein said fruit material essentially consists or consist of endocarp of the pyrenes of Canarium odontophyllum, wherein said fruit material preferably is essentially free or free of Canarium odontophyllum seeds, free of pulp and free of peel of Canarium odontophyllum fruits,
(a-ii) optionally drying the fruit material provided in step (a-i),
(a-iii) extracting the fruit material provided in step (a-i) or (a-ii) with a mixture essentially consisting or consisting of water and an alcohol having 1 to 3 carbon atoms or consisting of water and acetonitrile, preferably with a mixture of ethanol and water, wherein the total volume ratio (v/v) of said alcohol:water is in the range of 1:30 to 30:1, preferably in the range of 1:10 to 10:1, more preferably in the range of 1:5 to 5:1, even more preferably in the range of 1:2 to 2:1, yet more preferably in the range from 1.2:1 to 1:1.2, and most preferably in the range of 1:1,
(a-iv) partially or fully removing the alcohol having 1 to 3 carbon atoms of step (a-iii), preferably at a temperature below 80° C., more preferably in the range from 15 to 60° C., thereby obtaining an aqueous residue as extract,
(a-v) preferably mixing the extract obtained in step (a-iii) or the enriched extract obtained in step (a-iv) with one or more solid carrier substances, preferably one or more solid carrier substances selected from the group consisting of maltodextrins, silica, talc, lactose, sorbitol, mannitol, starches, gum acacia, calcium phosphate, orally acceptable stearate salts, preferably magnesium stearate, alginates, tragacanth, gelatins, calcium silicates, cellulose and cellulose derivatives, preferably amorphous cellulose, microcrystalline cellulose or methyl cellulose, polyvinylpyrrolidones, and propylhydroxybenzoates,
(a-vi) optionally drying the enriched extract obtained in step (a-iv) or the mixture obtained in step (a-v), preferably by spray-drying or freeze-drying.

Preferably, the total dry weight share of an extract according to the present invention is in the range from 20 to 100 wt. %, more preferably from 35 to 100 wt. %, particularly preferably from 50 to 100 wt. %, especially preferably from 55 to 98 wt. %, most preferably from 60 to 95 wt. %, in each case based on the total weight of the extract formulation.

The extract formulations according to the present invention or produced according to a method of the present invention may be used as such, in the form or pharmaceutical or nutraceutical compositions (the latter term including food additives) or in the form of functional food.

“Nutraceuticals”, “Functional Food”, or “Functional Food products” (sometimes also called “Foodsceuticals”, “Medicinal Food” or “Designer Food”) for use according to the present invention are defined as food products (including beverages) suitable for human consumption—the expression comprises any fresh or processed food having a health-promoting and/or disease-preventing property beyond the basic nutritional function of supplying nutrients, including food made from functional food ingredients or fortified with health-promoting additives, especially with effects in the modulation of phosphodiesterase dependent conditions.

The primary object of the present invention was to identify alternative active substances and substance mixtures that inhibit at least one phosphodiesterase (PDE), preferably phosphodiesterase dependent conditions. Especially the modulation of phosphodiesterase 5 (PDE5 or PDE-V) and phosphodiesterase 2 (PDE2 or PDE-II) dependent conditions in the human body. PDE5 or PDE2 inhibiting activity can induce vascular smooth muscle relaxation (PDE5) or improve of pulmonary conditions (PDE5), male and female sexual dysfunction (PDE5) or improve acute respiratory distress syndrome (PDE5) or enhance cognition (PDE2) in mammals

Additionally, said substances should preferably be naturally occurring compounds.

The functional food products or pharmaceutical products (compositions) according to the invention may be manufactured according to any suitable process, preferably ad-mixing an extract according to the present invention or produced according to a method of the present invention to a functional food product or at least one physiologically, nutraceutically or pharmaceutically, acceptable carrier.

Preferably, a functional food, pharmaceutical or nutraceutical composition comprising an extract or extract formulation according to the present invention, can be obtained by

(a) performing an extraction from Canarium odontophyllum fruit material in accordance with a method according to the present invention, or providing an extract or extract formulation according to the present invention,
(b) mixing the extract or extract formulation of step (a) as active ingredient in the preparation of the functional food product with the other constituents thereof or in order to obtain a functional food, pharmaceutical or nutraceutical composition with one or more carrier materials and/or one or more liquid solvents.

Further processing steps may precede and/or follow, such as drying (e.g. freeze-drying (lyophilization), spray-drying and evaporation), granulation, agglomeration, concentrating (e.g. to syrups, formed via concentration and/or with the aid of thickeners), pasteurizing, sterilizing, freezing, dissolving, dispersing, filtering, centrifuging, confectioning, and the like.

When an extract or extract formulation according to the present invention or an extract or extract formulation obtained according to a method of the present invention is added to a food product or pharmaceutical or nutraceutical, this also results in a functional food product or pharmaceutical or nutraceutical composition according to the invention.

Further additives may be included, such as vitamins, minerals, e.g. in the form of mineral salts, unsaturated fatty acids or oils or fats comprising them, other extracts, or the like.

The functional food products according to the invention may be of any food type. They may comprise one or more common food ingredients in addition to the food product, such as flavours, fragrances, sugars, fruit, minerals, vitamins, stabilizers, thickeners, dietary fibers, protein, amino acids or the like in appropriate amounts, or mixtures of two or more thereof, in accordance with the desired type of food product.

Examples of food products and thus of functional food products according to the invention are fruit or juice products, such as orange and grapefruit, tropical fruits, banana, apple, peach, blackberry, cranberry, plum, prune, apricot, cherry, peer, strawberry, marionberry, black currant, red currant, tomato, vegetable, e.g. carrot, or blueberry juice, soy-based beverages, or concentrates thereof, respectively; lemonades; extracts, e.g. coffee, tea, green tea; dairy type products, such as milk, dairy spreads, quark, cheese, cream cheese, custards, puddings, mousses, milk type drinks and yoghurt; frozen confectionary products, such as ice-cream, frozen yoghurt, sorbet, ice milk, frozen custard, water-ices, granitas and frozen fruit purees; baked goods, such as bread, cakes, biscuits, cookies or crackers; spreads, e.g. margarine, butter, peanut butter honey; snacks, e.g. chocolate bars, muesli bars; pasta products or other cereal products, such as muesli; ready-to-serve-dishes; frozen food; tinned food; syrups; oils, such as salad oil; sauces, such as salad dressings, mayonnaise; fillings; dips; chewing gums; sherbet; spices; cooking salt; instant drink powders, such as instant coffee, instant tee or instant cocoa powder; instant powders e.g. for pudding or other desserts; meat fish or fish or meat products, such as sausages, burgers, meat loafs, meatballs, meat extracts, canned or tinned fish or meat, meat vol-au-vent, meat or fish soup, meat or fish skewers, fish fingers; or the like.

One or more other customary additives may be present, such as flavours, fragrances or other additives, such as one or more selected from stabilizers, e.g. thickeners; coloring agents, such as edible pigments or food dyes; bulking agents, polyols, such as xylitol, mannitol, maltitol or the like; preservatives, such as sodium or potassium benzoate, sodium or calcium carbonate or other food grade preservatives; antioxidants, such as ascorbic acid, carotinoids, tocopherols or polyphenols; mono-, oligo- or polysaccharides, such as glucose, fructose, sucrose, soy-oligosaccharides, xylo-oligosaccharides, galacto-oligosacharides; other artificial or natural non- or low-caloric sweeteners, such as aspartame or acesulfame; bitterness blockers; acidifiers in the form of edible acids, such as citric acids, acetic acid, lactic acid, adipic acid; flavours, e.g. artificial or natural (e.g. botanical flavours); emulsifiers; thiols, e.g. allylicthiols; diluents, e.g. maltodextrose; wetting agents, e.g. glycerol; stabilizers; coatings; isotonic agents; absorption promoting or delaying agents; and/or the like.

An extract or extract formulation according to the invention or produced according to a method of the present invention can also be included in confectioned compositions to be added to foods including beverages, e.g. in the form of powders or granules, e.g. freeze-dried or spray-dried, concentrates, solutions, dispersions or other instant form, or the like.

In yet another aspect, the present invention relates to a pharmaceutical or nutraceutical composition, preferably a enterally, e.g. nasally or orally administrable composition, comprising an effective amount of an extract according to the present invention or produced according to a method of the present invention, and preferably additionally one or more physiologically (pharmacologically) acceptable carrier substances.

The pharmaceutical or nutraceutical compositions according to the present invention can be prepared in various forms, such as granules, tablets, pills, pellets, powders, syrups, solutions, dispersions, suppositories, capsules, suspensions, salves, lotions and the like. Pharmaceutical grade or food grade organic or inorganic carriers and/or diluents suitable for oral and topical use can be used to formulate compositions containing the therapeutically-active compounds. Diluents known in the art include aqueous media, vegetable and animal oils and fats or other carrier materials mentioned elsewhere herein. Stabilizing agents, wetting and emulsifying agents, salts for varying the osmotic pressure or buffers for securing an adequate pH value, and skin penetration enhancers can be used as auxiliary agents. The compositions may also include one or more of the following: carrier proteins such as serum albumin; buffers; fillers such as microcrystalline cellulose, lactose, corn and other starches; binding agents; sweeteners and other flavouring or fragrance agents; coloring agents; and polyethylene glycol. Those additives are well known in the art, and are used in a variety of formulations.

An extract or extract formulation according to the present invention or produced according to a method of the present invention may be formulated to be administered or may be administered alone or in combination with other active agents, preferably on or more other phosphodiesterase inhibitors.

An advantageous composition or combination or combination product (e.g. kit of parts or fixed combination) according to the invention additionally contains one or more further inhibitors of phosphodiesterase, preferably selected from the group of xanthines, preferably those described in US 2010/0285153 A1. Preferred xanthines are methyl xanthines, preferably selected from the group consisting of caffeine, theobromine and theophylline; the most preferred methyl xanthine in the sense of the present invention is caffeine. An alternatively preferred inhibitors of phosphodiesterase is aminophylline, a theophylline derivative.

A preferred composition according to the present invention additionally comprises one, two or more further ingredients selected from the group consisting of: preservatives, antimicrobial agents, antiinflammatory agents, antiirritants, antioxidants, chelating agents, moisture regulators, UV filters, fatty oils, fats, saturated fatty acids, mono- or polyunsaturated fatty acids, alpha-hydroxy acids, polyhydroxy-fatty acids, abrasives, binders, thickeners, buffers, dyestuffs, colorants, pigments, re-oiling (re-fatting) agents, emulsifiers, surfactants, detergents, extracts of algae or microalgae, vitamins and electrolytes.

A preferred composition according to the present invention is in a form selected from the group consisting of orally consumable granules, tablets, pills, capsules, pellets, syrups, powders, solutions, and dispersions.

Where “use” is mentioned, this especially refers to one or more of the following embodiments of the invention which can be inserted wherever use is mentioned:

(1) An extract or extract formulation according to the present invention for use in therapeutic (including prophylactic) treatment of a mammal, especially a human, for phosphodiesterase (PDE) dependent conditions, especially the modulation (preferably prophylactic and/or therapeutic treatment) of phosphodiesterase5 (PDE5 or PDE-V) and phosphodiesterase2 (PDE2 or PDE-II) dependent conditions in the human body. PDE5 or PDE2 inhibiting activity can be used in a prophlyctic and/or a therapeutic treatment to induce vascular smooth muscle relaxation (PDE5) or to improve pulmonary conditions (conditions meaning especially diseases but also the amelioration of a healthy status) (PDE5), in the prophylactic and/or therapeutic treatment of male and female sexual dysfunction (PDE5), or for the prophylactic and/or therapeutic treatment of (especially in order to improve it) acute respiratory distress syndrome (PDE5) or enhance cognition (PDE2) in mammals
(2) A pharmaceutical or nutraceutical composition comprising an extract according to the present invention as active ingredient together with a pharmaceutically acceptable diluent or carrier, especially for use in the therapeutic and/or prophylactic treatment mentioned under (1).
(2′) A pharmaceutical or nutraceutical composition for the treatment as mentioned under (1) comprising an extract or extract formulation according to the present invention, and a pharmaceutically acceptable diluent or carrier, as active ingredient supplement to a food.
(3) A functional food comprising an extract or extract formulation according to the present invention, as active ingredient for the treatment as mentioned under (1).
(4) A method for the treatment as mentioned under (1).
(5) A treatment of a subject, comprising administering a pharmaceutically or nutraceutically effective amount of an extract or extract formulation according to the present invention as active ingredient, especially to an individual in need thereof.
(6) The use of an extract or extract formulation according to the present invention as active ingredient for the manufacture of a medicament or nutraceutical or food supplement for the treatment mentioned under (1).
(7) A method or use as defined under (4), comprising co-administration, e.g. concomitantly or in sequence, especially in a jointly active way, of a therapeutically effective amount of an extract or extract formulation according to the present invention as active ingredient and a different pharmaceutically active compound and/or a pharmaceutically acceptable salt thereof, said different pharmaceutically active compound and/or salt thereof being especially for use in the treatment as mentioned under (1).
(8) A combination product (e.g. in the form of a kit of parts) comprising a therapeutically effective amount of an extract or an extract formulation according to the present invention as active ingredient, and a different pharmaceutically active compound and/or a pharmaceutically acceptable salt thereof, said second pharmaceutically active compound being especially for use or of use in the treatment mentioned under (1).

The pharmaceutical or nutraceutical preparations (compositions) may be sterilized and/or may contain carrier materials or adjuvants such as preservatives, stabilizers, binders, disintegrants, wetting agents, skin or mucuous membrane penetration enhancers, emulsifiers, salts for varying the osmotic pressure and/or buffers, or other ingredients, excipients or carrier materials known in the art.

The extracts or extract formulations or compositions can be administered by a variety of routes including oral, rectal, topical, transdermal, subcutaneous, intravenous, intramuscular, and intranasal, preferably they are administered enterally, e.g. nasally or especially orally.

The extracts or extract formulations according to the present invention or produced according to a method of the present invention are preferably formulated as compositions prior to administration. Therefore, another embodiment of the present invention is a pharmaceutical composition comprising an effective amount of an extract or extract formulation according to the present invention or produced according to a method of the present invention and a pharmaceutically acceptable carrier, diluent or excipient therefore.

By physiologically, preferably pharmaceutically and/or nutraceutically, acceptable it is meant that the carrier, diluent or excipient is compatible with the other ingredients of the formulation and not be deleterious to the recipient thereof.

The present compositions may be prepared by known procedures using well known and readily available further ingredients. In making the compositions of the present invention, the active ingredient will usually be admixed with a carrier, or diluted by a carrier, or enclosed within a carrier which may be in the form of a capsule, sachet, paper or other container.

Examples of suitable carriers, excipients, and diluents for compositions of the present invention may, alternatively to already mentioned definitions, be selected from one or more of lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methylhydroxybenzoates, propylhydroxybenzoates, talc, magnesium stearate and mineral oil. The formulations may additionally include lubricating agents, wetting agents, sweetening agents, flavoring agents, and the like. The compositions of the invention may be formulated so as to provide quick, sustained or delayed release of the active ingredient after administration to the patient by employing procedures well known in the art.

In a preferred embodiment, the compositions are preferably formulated in a unit dosage from. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

A nutraceutical or pharmaceutical composition according to the invention is e.g. in the form of unit dosage forms, such as tablets, capsules or sachets, that contain e.g. 1 mg to 2 g, for example 5 mg to 1 g, of the extract obtainable according to the invention.

The dosage in both nutraceutical or pharmaceutical use/compositions typically is such that the amount of the extract administered to a mammal, e.g. a human, is such that it is effective in modulating the targets mentioned above and below, or preferably a daily dose of about 0.01 to 100 g, e.g. 0.1 to 5 g is administered to a person with a weight of 70 kg per day in one or more, e.g. 1 to 3, dosages (children or persons with differing weights receive a correspondingly modified dosage).

The present invention is further illustrated by the following examples. The specific examples which follow illustrate the methods in which the compositions of the present invention may be prepared, components therein and their use, as well as other embodiments of the invention, but are not to be construed as limiting the invention in scope.

### EXAMPLE 1

### Plant Material Used

Whole fresh fruits from Canarium odontophyllum (harvested in Malaysia) at the mature stage were used in the following experiments.

1500 g fresh fruits from Canarium odontophyllum were manually separated into peel/pulp, endocarp (A) (ref. FIG. 1) and seed (B) (ref. FIG. 1), followed by freeze-drying of the isolated materials. The yields for the lyophilized materials are given in table 1.

The dry endocarp (A) was ground into a powder by first using a cutting mill followed by a lab centrifugal mill.

TABLE 1
Yields of lyophilized fruit part materials obtained
from 1500 g fresh Canarium odontophyllum fruits

| Fruit part | Amount [g] |
|---|---|
| peel/pulp | 442 |
| endocarp (A) (ref. FIG. 1) | 420 |
| seeds (B) (ref. FIG. 1) | 83 |

### EXAMPLE 2

### Extraction and Extract

### Preparation of a Crude Aqueous or Ethanolic Extract from Endocarp and Subsequent Enrichment

### Extract Generation

In each case 20 g milled endocarp material were extracted with water or water:ethanol mixture:

Extraction 1: 100% water; one time; 200 ml; 95° C.; stirring; 60 minutes
Extraction 2: 30% ethanol; two times; each 100 ml; max. 40° C.; ultrasound; 30 minutes
Extraction 3: 50% ethanol; two times; each 100 ml; max. 40° C.; ultrasound; 30 minutes
Extraction 4: 70% ethanol; two times; each 100 ml; max. 40° C.; ultrasound; 30 minutes
Extraction 5: 90% ethanol; two times; each 100 ml; max. 40° C.; ultrasound; 30 minutes

### Extraction 1—Hot Water Extraction

20 g of milled plant material were extracted with 200 ml water under reflux and stirring for 60 minutes. The resulting suspension was filtered and the filtrate was split into two aliquots. In order to yield the crude dry extract, one quarter of the filtrate was directly evaporated under reduced pressure to dryness (rotary evaporator, max. water bath temperature 40° C.). For crude extract yields refer to table 2.

The remaining three quarters of the filtrate were further processed to the enriched extract as follows: Water was added to a final volume of 200 ml and subsequently extracted three times with 200 ml ethyl acetate each in a liquid/liquid separation. The organic phases were won and the combined organic phases were then evaporated to dryness under reduced pressure at a maximum temperature of 40° C. For the enriched extract yields refer to table 2.

### Extraction 2 to 5—Ethanolic Extraction

20 g of milled plant material each were extracted for 30 minutes applying ultrasound at a maximum temperature of 40° C. The resulting suspension was filtered and the residual endocarp material was extracted a second time under the same conditions.

The combined filtrates were split into two aliquots. In order to yield the crude extract, one quarter was directly evaporated under reduced pressure to dryness (rotary evaporator, max. water bath temperature 40° C.). For extraction yields refer to table 2. The remaining three quarters of the filtrate were further processes to the enriched extract as follows: The filtrates were concentrated under reduced pressure at a maximum temperature of 40° C., thereby removing essentially all the ethanol. To the residual water phase further water was added to a final volume of 200 ml. The resulting water phase was subsequently extracted three times with 200 ml ethyl acetate each in a liquid/liquid separation. The combined organic phases were then evaporated to dryness under reduced pressure at a maximum temperature of 40° C. For the enriched extract yields thus obtained refer to table 2.

All crude extracts as well as the corresponding enriched ethylacetate extracts were used in the biological activity test experiments described in Example 3 and 4 below.

TABLE 2
Yields of prepared Canarium odontophyllum endocarp extracts

| | Extraction | | | |
|---|---|---|---|---|
| Experiment | solvent | Sample-Code | Extract type | Yield [mg] |
| Extraction 1 | 100% water | EXT-01-01-01 | crude | 111.20 |
| | | EXT-01-01-02 | enriched | 83.10 |
| | | | EE phase | |
| Extraction 2 | 30% EtOH | EXT-01-02-01 | crude | 85.00 |
| | | EXT-01-02-02 | enriched | 102.10 |
| | | | EE phase | |
| Extraction 3 | 50% EtOH | EXT-01-03-01 | crude | 123.90 |
| | | EXT-01-03-02 | enriched | 129.50 |
| | | | EE phase | |
| Extraction 4 | 70% EtOH | EXT-01-04-01 | crude | 113.40 |
| | | EXT-01-04-02 | enriched | 154.10 |
| | | | EE phase | |
| Extraction 5 | 90% EtOH | EXT-01-05-01 | crude | 52.00 |
| | | EXT-01-05-02 | enriched | 57.90 |
| | | | EE phase | |

Note the data stem from the fact that one quarter of the crude extract was used for determining the amount therein, while three quarters were further processed as indicated.

### EXAMPLE 3

### Human Phosphodiesterase-5 (PDE5 or -2 (PDE2A1)) Activity Testing Based on AlphaScreen® Assay Technology

Phosphodiesterase inhibitory activity of the test items prepared according to the procedure in example 2 and outlined in table 2 were measured using the AlphaScreen® assay technology. Materials and methods were according to technical instructions given by Perkin Elmer (www.perkinelmer.com; “Application note “AlphaScreen cGMP Phosphodiesterase Assay Using AlphaScreen cGMP Supplement with Anti-cGMP Antibody”; Authors: Martin Boissonneault, Christian Fafard, Joe Trometer, Chantal Illy, Stéphane Parent, and Roger Bossé; PerkinElmer Life and Analytical Sciences, 710 Bridgeport Avenue, Shelton, Conn. 06484 USA).

The AlphaScreen cGMP PDE assay used PerkinElmer's AlphaScreen cGMP supplement with antibody (cat. no. 6760308M or 6760308R) which consists of a biotinylated cGMP derivative and an anti-cGMP antibody. The assay also requires the AlphaScreen Protein A detection kit (PerkinElmer cat. no. 6760617), composed of Protein A acceptor beads and Streptavidin donor beads. The human recombinant PDE5A (N-terminal GST, cat no 60050) and PDE2A1 (N-terminal FLAG tag, cat no 0021) were purchased from BPS Bioscience, San Diego, United States. 4-{[3′,4′-(Methylenedioxy)benzyl]amino}-6-methoxyquinazoline was purchased from Calbiochem® (cat. no.524715). cGMP (cat no.G6129), Zaprinast (cat no. Z0878; reference inhibitor for PDE5) and EHNA.HCl [erythro-9-(2-hydroxy-3-nonyl)adenine) hydrochloride](cat no E114; reference inhibitor for PDE2A1) were obtained from Sigma-Aldrich™.

### Chemicals Used

Hepes (Sigma, Schnelldorf, Germany, Cat.# H4034)
MgCl2×6 H₂O (Acros Organics, New Jersey, USA, Cat.#197530010)
Bovine Serum Albumin (Sigma, Schnelldorf, Germany, Cat.# A7030)
NaCl (Fisher BioReagents, New Jersey, USA, Cat.# BP358-1)
EDTA (Ethylenediaminetetraacetic acid—Dihydrate) (Merck, Darmstadt, Germany, order. No. 1.12029.0100)
Tween-20 (Sigma, Schnelldorf, Germany, Cat.# P2287)
IgG Detection Kit (Perkin Elmar, Rodgau, Germany; Cat.#6760617C)
   cGMP supplement (Cat.#6760306M)
   b-cGMP supplement (Cat.#6760002)
   anti-cGMP-Antibody (Cat.#676308M)
   Control Buffer (10×),
DMSO (Dimethylsulfoxid) (Sigma-Aldrich, Schnelldorf, Germany, Cat#34869)

### Technical Equipment/Material

PHEAstar multi titer plate reader (BMG LABTECH GmbH, Ortenberg; Germany)
OptiPlate-384, White Opaque MTP (Perkin Elmar, Rodgau, Germany, Cat#6007299)

### Method

To perform the cGMP PDE assay, inhibitors tested were diluted in the PDE reaction buffer supplemented with 400 nM cGMP. The PDE stock solution was diluted to 1 unit/μL in the PDE reaction buffer (25 mM Hepes pH 7.4, 2.5 mMMgCl2, 0.1% BSA). The 1.25 μM AlphaScreen cGMP Supplement stock solution was diluted to 2.5 nM in the Stop/Detection buffer (25 mM Hepes pH 7.4, 100 mMNaCl, 0.1% Tween-20, 25 mM EDTA). The detection mix was prepared by diluting the AlphaScreen Streptavidin-Donor beads and Protein A-Acceptor beads to 50 μg/mL, and the anti-cGMP antibody (1:2000) in the Assay buffer. The detection mix was allowed to pre-incubate for 30 minutes at room temperature.

The cGMP PDE assay was per-formed following these steps:

1—Add 5 μL of inhibitor* * Please note that for compound screening purposes, adding 0.54 of test compounds to 4.5 μL of cGMP substrate solution prepared in the PDE reaction Buffer is recommended. If the test compounds are diluted in 100% DMSO, this will bring DMSO to a safe 2% final concentration in the assay.
2—Add 5 μL of PDE (5U per well)
3—Incubate 120 minutes at 23° C. for enzymatic cleavage of unlabeled cGMP
4—Add 5 μL of the AlphaScreen cGMP Supplement (1 nM final)
5—Add 10 μL Detection mix (anti-cGMP antibody/Streptavidin-Donor beads (20 μg/mL final)/Protein A-Acceptor beads (20 μg/mL final))
6—Incubate the plate for 1 hour at room temperature in the dark and detect the AlphaScreen signal using the microplate analyzer PHERAstar reader.

### PDE-5 Activities

All extracts showed inhibition of PDE5. The EtOH extracts show higher activity compared to the water extract. Furthermore, the EE phases are stronger compared to the crude extracts with 1050 values below 10 μg/ml.

TABLE 3
Inhibition of PDE5 by Canarium odontophyllum endocarp extracts

| | | Concentration | | Concentration | | Concentration | |
|---|---|---|---|---|---|---|---|
| | | 100 μg/ml | | 30 μg/ml | | 10 μg/ml | |
| Sample | | % | % SD | % | % SD | % | % SD |
| code | Extract type | inhibition | (n = 3) | inhibition | (n = 3) | inhibition | (n = 3) |
| EXT-01- | water; | 69 | 4 | 11 | 11 | 0 | 0 |
| 01 | crude extract | | | | | | |
| EXT-01- | water; | 95 | 3 | 55 | 5 | 0 | 5 |
| 02 | EE phase | | | | | | |
| EXT-02- | 30% EtOH; | 81 | 7 | 58 | 5 | 6 | 8 |
| 01 | crude extract | | | | | | |
| EXT-02- | 30% EtOH; | 100 | 2 | 87 | 10 | 58 | 3 |
| 02 | EE phase | | | | | | |
| EXT-03- | 50% EtOH; | 100 | 3 | 96 | 3 | 59 | 4 |
| 01 | crude extract | | | | | | |
| EXT-03- | 50% EtOH; | 100 | 2 | 100 | 3 | 70 | 3 |
| 02 | EE phase | | | | | | |
| EXT-04- | 70% EtOH; | 100 | 3 | 86 | 2 | 53 | 8 |
| 01 | crude extract | | | | | | |
| EXT-04- | 70% EtOH; | 100 | 3 | 99 | 2 | 66 | 4 |
| 02 | EE phase | | | | | | |
| EXT-05- | 90% EtOH; | 98 | 4 | 72 | 7 | 24 | 8 |
| 01 | crude extract | | | | | | |
| EXT-05- | 90% EtOH; | 100 | 2 | 97 | 7 | 66 | 6 |
| 02 | EE phase | | | | | | |
| Zaprinast | reference | 88 | 2 | | | | |
| | at 19 μM | | | | | | |
| Zaprinast | reference | 81 | 3 | | | | |
| | at 5 μM | | | | | | |
| Zaprinast | reference | 57 | 5 | | | | |
| | at 1 μM | | | | | | |

SD = standard deviation
The inhibition of PDE5 was tested at concentrations of 100, 30, and 10 μg/ml.

### PDE2 Activities

All extracts show strong inhibition of PDE2.

TABLE 4
Inhibition of PDE-2 by Canarium odontophyllum endocarp extracts

| | | Concentration | | Concentration | | Concentration | |
|---|---|---|---|---|---|---|---|
| | | 100 μg/ml | | 30 μg/ml | | 10 μg/ml | |
| Sample | | % | % SD | % | % SD | % | % SD |
| code | Extract type | inhibition | (n = 3) | inhibition | (n = 3) | inhibition | (n = 3) |
| EXT-02- | 30% EtOH; | 100 | 1 | 98 | 4 | 50 | 5 |
| 01 | Crude extract | | | | | | |
| EXT-02- | 30% EtOH; | 100 | 2 | 100 | 7 | 67 | 7 |
| 02 | EE-Phase | | | | | | |
| EXT-03- | 50% EtOH; | 100 | 4 | 100 | 5 | 83 | 8 |
| 01 | Crude extract | | | | | | |
| EXT-03- | 50% EtOH; | 78 | 19 | 100 | 14 | 72 | 7 |
| 02 | EE-Phase | | | | | | |
| EXT-04- | 70% EtOH; | 100 | 1 | 94 | 1 | 47 | 11 |
| 01 | Crude extract | | | | | | |
| EXT-04- | 70% EtOH; | 100 | 8 | 100 | 4 | 89 | 3 |
| 02 | EE-Phase | | | | | | |
| EHNA | Reference | 98 | 1 | | | | |
| | at 10 μM | | | | | | |
| EHNA | Reference | 73 | 6 | | | | |
| | at 5 μM | | | | | | |
| EHNA | Reference | 33 | 17 | | | | |
| | at 1 μM | | | | | | |

SD = standard deviation

### EXAMPLE 4

### Ex Vivo in Tissue Testing—Aorta Ring Assay

### a) Extract Generation

200 g endocarp material according Example 1 were ground into a powder by first using a cutting mill followed by using a lab centrifugal mill. The milled material was extracted twice with 1000 ml water:ethanol (1:1) at 40° C. for 30 minutes applying ultrasound. The remaining plant material was removed by filtration and the crude extract solution was separated for further processing. In order to yield the crude extract, one quarter (400 ml) of the extract solution was directly evaporated under reduced pressure to dryness (rotary evaporator, max. water bath temperature 40° C.). From the remaining ¾ of the extract solution (1200 ml) the ethanol was removed by evaporation (rotary evaporator, max. water bath temperature 40° C.). The remaining water phase was filled up to 500 ml and subsequently enriched by liquid/liquid separation, three times with 500 ml ethylacetate. The combined ethylacetate phases from this liq./liq.-separation were evaporated under reduced pressure to dryness. For extraction yields refer to Table 5.

TABLE 5
Prepared Canarium odontophyllum endocarp
extracts with corresponding yields.

| Sample code | Phase | Yield [mg] |
|---|---|---|
| EXT-06-01 | 50% ethanolic crude extract | 1873.5 |
| EXT-06-02 | ethylacetate phase | 1033.1 |

The 50% ethanolic crude extract (EXT-06-01) as well as the corresponding ethylacetate phase (EXT-06-02) was tested in an ex vivo in tissue assay (aorta ring assay).

### b) Biological Testing and Results (Rat Aortic Ring Assay—Ex Vivo in Tissue Testing)

An ex vivo in tissue assay was conducted in order to determine the reduction of Norepinephrine induced contraction of rat aorta tissue by Canarium odontophyllum endocarp extracts according example 4a).

The assay was conducted by Ricerca Bioscience (Ricerca Biosciences, LLC Pharmacology Laboratories; 158 Li-Teh Road, Peitou; Taipei, Taiwan 112; Taiwan R.O.C.; Assay catalogue number 412050).

An endothelial denuded aortic ring obtained from Wistar derived male or female rats weighing 275+/−25 g and sacrificed by CO₂ overexposure was used. The tissue was placed under 2 g tension in a 10 mL bath containing Krebs solution pH 7.4 at 37° C. and submaximal tonic isometrically recorded contraction was induced by norepinephrine (1 μM). The extracts were then administered (100, 30, 10, 3 μg/ml) to test for relaxation. 1 μM Acetylcholine was used as reference and 100% control as it is a known relaxant in this assay. The activity data for the 50% ethanolic crude extract and the corresponding enriched extract are given in table 6.

Literature: Komas N. et al., Br. J Pharmacol, 1991, Endothelium-dependent and independent relaxation of the rat aorta by cyclic nucleotide phosphodiesterase inhibitors

TABLE 6
Experimental results for Canarium odontophyllum
endocarp extracts

| Sample code | Phase | Conc. [μg/ml] | Activity* [%] |
|---|---|---|---|
| EXT-06-01 | 50% ethanolic crude | 100 | 125 |
| | extract | 30 | 92 |
| | | 10 | 45 |
| | | 3 | 20 |
| EXT-06-02 | ethylacetate phase | 100 | 105 |
| | | 30 | 88 |
| | | 10 | 70 |
| | | 3 | 21 |

*100% = the relaxation induced by 1 μM Acetylcholine

Both extracts, EXT-06-01 (50% ethanolic crude extract) and EXT-06-02 (ethylacetate phase from the crude extract) showed significant dose dependent activities in the aorta ring assay at concentrations from 3 to 100 μg/ml.

1. A method for producing an extract or extract formulation of the fruit of Canarium odontophyllum comprising:
(i) providing fruit material from Canarium odontophyllum, wherein said fruit material essentially consists of the endocarp of the seeds of Canarium odontophyllum,
(ii) extracting the fruit material provided in step (i) with a mixture consisting essentially of water and an alcohol having 1 to 3 carbon atoms or consisting of water and acetonitrile.

2. The method according to claim 1, wherein the fruit material provided in step (i) comprises 85 wt. % or more of woody endocarp of Canarium odontophyllum.

3. The method according to claim 2, wherein the fruit material provided in step (i) comprises less than 15 wt. % of seeds of Canarium odontophyllum.

4. The method according to claim 3, wherein the ratio by weight of the total amount of seedless woody endocarp of Canarium odontophyllum to the total amount of seeds of Canarium odontophyllum is greater than 5.1:1.

5. An extract or extract formulation obtainable by a method according to claim 1.

6. Extract or extract formulation obtainable by a method comprising or consisting of the following steps:
(a-i) providing fruit material from Canarium odontophyllum, wherein said fruit material consists essentially of the endocarp of the fruits of Canarium odontophyllum,
(a-ii) optionally drying the fruit material provided in step (a-i),
(a-iii) extracting the fruit material provided in step (i) or (i-a) with a mixture essentially consisting or consisting of water and an alcohol having 1 to 3 carbon atoms or consisting of water and acetonitrile, preferably with a mixture of ethanol and water, wherein the total volume ratio (v/v) of said alcohol:water is in the range of 1:30 to 30:1, preferably in the range of 1:10 to 10:1, more preferably in the range of 1:5 to 5:1, even more preferably in the range of 1:2 to 2:1, yet more preferably in the range from 1.2:1 to 1:1.2, and most preferably in the range of 1:1,
(a-iv) optionally partially or fully removing the alcohol having 1 to 3 carbon atoms or acetonitrile of step (ii), preferably at a temperature below 80° C., more preferably in the range from 15 to 60° C., thereby obtaining an aqueous residue, and
optionally adding an organic solvent of medium polarity to said aqueous residue and extracting the aqueous residue with said organic solvent or a supercritical solvent, such as supercritical carbon dioxide wherein the organic solvent is preferably selected from the group consisting of n-butanol, iso-butanol, 2-butanone, 3-pentanone, methyl n-propyl ketone, methyl iso-propyl ketone, methyl isobutyl ketone, methyl isoamyl ketone, methyl-t-butyl ether, methyl acetate, ethyl acetate, propyl acetate, butyl acetate, pentyl acetate, ethyl propanoate and ethyl butanoate or a mixture thereof, and subsequently separating the organic layer comprising the organic solvent of medium polarity,
thereby obtaining an enriched extract,
(a-v) preferably mixing the extract obtained in step (ii) or the enriched extract obtained in step (iii) with one or more solid carrier substances, preferably one or more solid carrier substances selected from the group consisting of maltodextrins, silica, talc, lactose, sorbitol, mannitol, dextrose, sucrose, starches, gum acacia, calcium phosphate, orally acceptable stearate salts, preferably magnesium stearate, alginates, tragacanth, gelatins, calcium silicates, cellulose and cellulose derivatives, preferably amorphous cellulose, microcrystalline cellulose or methyl cellulose, polyvinylpyrrolidones, and propylhydroxybenzoates, and
(a-vi) optionally drying the enriched extract obtained in step (a-iv) or the mixture obtained in step (a-v), preferably by spray-drying or freeze-drying.

7. A method of inhibiting phosphodiesterase activity comprising administering an effective amount of an extract of fruit material of the fruit of Canarium odontophyllum, wherein the fruit material comprises one or more pyrenes.

8. The method according to claim 7, wherein the fruit material from Canarium odontophyllum comprises 85 wt. % or more of the woody endocarp of fruits of Canarium odontophyllum and less than 15 wt. % of seeds of Canarium odontophyllum, based on the total amount of fruit material.

9. The method according to claim 7, further including prophylactic and/or therapeutic treatment of conditions related to the modulation of phophodiesterase 5 (PDE5 or PDE-V) and phosphodiesterase 2 (PDE2 or PDE-II) in the mammal body, especially in the human body, especially in a prophylactic and/or therapeutic treatment to induce vascular smooth muscle relaxation (PDE5) or to improve pulmonary conditions (PDE5), or prophylactic and/or therapeutic treatment in the case of male and female sexual dysfunction (PDE5), or for the prophylactic and/or therapeutic treatment of acute respiratory distress syndrome (PDE5) or in order to enhance cognition (PDE2) in mammals, preferably in human beings.

10. A pharmaceutical composition, preferably an orally administrable pharmaceutical composition, comprising
an effective amount of an extract obtained by a method according to claim 1 for use in the prophylactic and/or therapeutic treatment of conditions related to the modulation of phophodiesterase 5 (PDE5 or PDE-V) and phosphodiesterase 2 (PDE2 or PDE-II) in the mammal body, especially in the human body, especially in a prophylactic and/or therapeutic treatment to induce vascular smooth muscle relaxation (PDE5) or to improve pulmonary conditions (PDE5), or prophylactic and/or therapeutic treatment in the case of male and female sexual dysfunction (PDE5), or for the prophylactic and/or therapeutic treatment of acute respiratory distress syndrome (PDE5) or in order to enhance cognition (PDE2) in mammals, preferably in human beings.

11. The pharmaceutical composition according to claim 10, further comprising one or more additional pharmacologically active compound preferably selected from the group of
(i) phosphodiesterase inhibitors,
(ii) phosphodiesterase inhibitors 5, and
(iii) phosphodiesterase inhibitors 2.

12. The pharmaceutical composition according to claim 11 wherein, in each case based on the total weight of the composition,
the total quantity of phosphodiesterase inhibitors is in the range of from 0.005-10% by weight, preferably in the range of from 0.05-5% by weight and more preferably in the range of from 0.5-2.5% by weight.

13. The composition according to claim 10, wherein said composition is in a form selected from the group consisting of orally consumable granules, tablets, pills, capsules, pellets, syrups, powders, solutions, and dispersions.

14. The method according to claim 1, further comprising:
(i-a) drying the fruit material provided in step (i) prior to step (ii).

15. The method according to claim 1, further comprising:
(iii) partially or fully removing the alcohol having 1 to 3 carbon atoms or the acetonitrile of step (ii) to yield an aqueous residue.

16. The method according to claim 15, further comprising:
adding an organic solvent of medium polarity to said aqueous residue and extracting the aqueous residue with said organic solvent or a supercritical solvent, such as supercritical carbon dioxide, thereby obtaining an enriched extract.

17. The method according to claim 16, further comprising:
(iv) mixing the extract obtained in step (ii) or the enriched extract obtained in step (iii) with one or more solid carrier substances.

18. The method according to claim 17, further comprising:
(v) drying the enriched extract obtained in step (iii) or the mixture obtained in step (iv), preferably by spray-drying or freeze-drying.

19. The method according to claim 1, wherein the fruit material provided in step (i) comprises 90 wt. % or more of woody endocarp of Canarium odontophyllum and less than 12 wt. % of seeds of Canarium odontophyllum.

20. The extract or extract formulation according to claim 5 showing vasodilator activity, including at least PDE 5 or PDE 2 inhibition activity.
